(19) United States
(12) Patent Application Publication
(10) Pub. No.: US 2004/0171824 A1
(43) Pub. Date: Sep. 2, 2004

(54) A Lac operator-repressor system

(76) Inventors: Heidi Jean Scrable, Crozet, VA (US); CAROLYN ANNE CRONIN, CHARLOTTESVILLE, VA (US)

Correspondence Address: UNIVERSITY OF VIRGINIA PATENT FOUNDATION, 1224 WEST MAIN STREET, SUITE 1-110, CHARLOTTESVILLE, VA, 22903, US

(21) Appl. No.: 10/469,881

(22) PCT Filed: Mar. 5, 2002
(86) PCT No.: PCT/US02/06468

Related U.S. Application Data
(60) Provisional application No. 60/273,480, filed on Mar. 5, 2001.
(60) Provisional application No. 60/281,322, filed on Apr. 4, 2001.

(51) Int. Cl. C12Q 1/68; C07H 21/04; C07K 14/705; C12N 5/06

(52) U.S. Cl. 536/23.5; 530/350; 435/6; 435/69.1; 435/320.1; 435/325

(57) ABSTRACT

The present invention relates to a system that allows the specific regulation of a gene in a eukaryotic cell. The system comprises a novel repressor gene construct (shown in FIG. 1), wherein the construct comprises a promoter (stippled box) operably linked to a rabbit β-globin intron2/exon3 sequence (open box) which is operably linked to a modified lac repressor coding region which is operably linked to rabbit β-globin 3′ untranslated sequences (solid bar). The modified lac repressor coding region is made up of segments that are identical to the wild type bacterial sequence (crosshatched box), and segments that have been reencoded to use mammalian codons (striped box).

### RELATED APPLICATION

This application claims priority under 35 USC §199(e) to U.S. Provisional Application Serial Nos. 60/273,480, filed Mar. 5, 2001, and 60/281,322, filed Apr. 4, 2001, the disclosures of which are incorporated herein.

### US GOVERNMENT RIGHTS

This invention was made with United States Government support under Grant No. NCRR 11102 and MH 12406, awarded by National Institutes of Health. The United States Government has certain rights in the invention.

### FIELD OF THE INVENTION

The present invention is directed to a system for regulating the expression of a gene in an animal. The system comprises a novel gene construct that encodes a repressor protein wherein the repressor functions to bind to the operator region of a recombinant gene and inhibit transcription of therecombinant gene in an animal. Expression of the gene is increased upon the administration of an exogenous inducer agent to the animal, wherein the inducing agent causes the removal of the repressor from the operator.

### BACKGROUND OF THE INVENTION

Jacob and Monod described the system of structural and regulatory elements that make up the lac operon of E. coli. This set of genes is coordinately regulated by lactose, a metabolite used by bacteria as an energy source when it is present in their environment. The regulatory components of the system are the lac repressor and its DNA binding sequence, the lac operator. These two elements control the transcription of the rest of the genes in the lac operon that encode enzymes necessary for lactose metabolism. In the absence of lactose, the lac repressor occupies the lac operators, altering the structure of the promoter in the region of the RNA polymerase binding site, and preventing transcription. Lactose causes a conformational change in the repressor and it vacates the operators, allowing RNA polymerase to gain access to the promoter and initiate transcription.

Hu and Davidson (Hu and Davidson, Cell 48(4), 555-566,1987) were the first to use lac elements to control reporter gene expression and activity in mammalian cells. They modified the bacterial GTG initiator codon of lacIq to ATG and used the Rous sarcoma virus LTR to drive lacI expression. They showed that mouse L-cells stably transfected with this lacI expression vector produced sufficient lac repressor protein to control the expression and activity of an MSV-CAT reporter gene with lac operators inserted into the promoter. The lactose analog, isopropyl-β-D-thiogalactoside (IPTG), caused a marked de-repression of CAT activity in mouse L-cells demonstrating that the system was also reversible. This result was extended by Figge et al. (Figge et al., Cell 52(5), 713-722, 1988) to stably integrated regulatable reporter genes in monkey cell lines.

It has long been recognized that a system that would allow similar control over the activity of genes in animals, such as the mouse, would be extremely useful. In 1997 it was reported that transgenes containing the bacterial coding sequence for the lac repressor downstream of the β-actin promoter were heavily methylated and only transcribed in the testis of transgenic mice. Methylation and silencing in mice was also observed when the bacterial lac repressor sequence was downstream of the F9-1 polyoma promoter. In an attempt to reactivate silenced transgenes, the primary DNA sequence of the bacterial lacI gene was changed to resemble a mammalian coding sequence more closely and still code for the same amino acid sequence. Although this synlacI gene was widely transcribed, it did not produce a functional product. To achieve the successful transfer of a fully functional lac operator-repressor system to the mouse, extensive modifications to the synlacI gene were required, as described herein.

The present invention is directed to a system that uses elements from the lac operon of E. coli for controlling phenotype in an animal. More particularly, the present invention is directed to a regulatory system for controlling the expression of recombinant genes in animals, including mammalian species. One important component of this regulatory system is a lac repressor transgene that expresses functional levels of repressor protein in the transgenic mouse. Although others have attempted to utilize prokaryotic repressor proteins in eukaryotic cells to control the expression of genes, none have succeeded in preparing a system that uses the lac repressor to regulate expression in mammals in vivo.

For example, there is a reversible system based on the tet operon of E. coli that is commercially available. However, when Gossen and Bujard adapted the tet system for use in mammalian cells, they converted the tet repressor into a tet transactivator. This conversion resulted from the fusion of the tet repressor with the activating domain of the herpes simplex virus VP16 transcriptional activator, and thus it is necessary to permanently couple the tet operator to a viral promoter (Gossen and Bujard, PNAS 89(12), 5547-51, 1992) for the system to work. Binding of repressor to the operator serves only to align the VP16 fusion partner with its specific binding site in the viral promoter, and it is the binding of VP16 to the viral promoter that activates transcription. This dependence of the tet system on viral promoter elements limits its applicability in the mouse, where non-mammalian promoters very frequently lead to erratic expression of downstream coding sequences. Low level leakiness and heterogeneous expression (Redfern et al. PNAS, 97(9), 4826-31, 1999) have been problems with use of the minimal CMV promoter, and the VP16 activating domain has been found to be toxic to cells. Furthermore, while U.S. Pat. No. 5,589,392 discloses the use of the lac repressor in an inducible mammalian expression system, this system also uses viral promoters and fails to produce adequate levels of lac repressor in mice.

The present invention describes nucleic acid constructs and methods that eliminate the necessity of using viral promoters or viral DNA binding proteins in a prokaryotic-based regulatory system. This lends the system a particularly strong element of predictability that other prokaryotic-based systems cannot match. Another significant advantage is that, in addition to being able to regulate a mammalian promoter as part of a transgene, the lac system holds the promise of endogenous gene regulation. By inserting lac operators into an endogenous promoter (or elsewhere in a gene) by homologous recombination, it should be possible to gain control over resident genes to create mouse models of disease and to elucidate gene function in their natural context.

### SUMMARY OF THE INVENTION

The present invention is directed to a novel repressor protein gene construct, derived from the E. coli lac repressor gene. The present invention also encompasses an inducible bacterial expression system and the use of that system for regulating the expression of genes in vivo in an animal. The system comprises two main elements, the first being a novel gene construct for expressing the lac repressor protein, and the second being a gene that is operably linked to an operator region. In this system, the repressor protein binds to the operator in the absence of the inducer agent to prevent transcription of the gene. Subsequent addition of the inducer causes the release of the repressor, allowing expression of the gene.

### BRIEF DESCRIPTION OF THE DRAWINGS

FIG. 1. Schematic representation of the lacI^{R} transgene. The gene construct comprises a promoter (stippled box) operably linked to a rabbit β-globin intron2/exon3 sequence (open box) which is operably linked to a modified lac repressor coding region which is operably linked to rabbit β-globin 3′ untranslated sequences (solid bar). The modified lac repressor coding region is made up of segments that are identical to the wild type bacterial sequence (crosshatched box), and segments that have been reencoded to use mammalian codons (striped box).

FIG. 2. Regulatable Tyr^{lacO} transgene. Three lac operators have been introduced into the murine tyrosinase promoter. The primary operator was centered just downstream of the start of transcription by changing the endogenous promoter sequence; two additional operators were inserted 176 bp and 526 bp upstream. The modified promoter drives expression of the wild type murine tyrosinase cDNA.

FIG. 3 Diagram of the lacO-promoter trap vector. The lac OCR elements are indicated with two vertical stippled rectangles (the operator sequences) separated by a black rectangle (the 150 bp stuffer). Each OCR is separated by a 400 bp fragment from the rabbit β-globin IVS2 (striped rectangle). loxP sites are indicated with ovals. The IRES-GFPneo cassette (crosshatched rectangle) with associated 3′splice site (3′ spl) and poly(A) addition site (pA) are indicated

### DETAILED DESCRIPTION OF THE INVENTION

Definitions

In describing and claiming the invention, the following terminology will be used in accordance with the definitions set forth below.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH 2—S(O)2NR—) linkage, a urea (—NHC(O)NH—) linkage, a —CH2 -secondary amine linkage, or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a —NHC(O)NHR group where R and R1 are hydrogen or C1-C4 alkyl with the proviso that R and R1 are not both hydrogen;

3. peptides wherein the C terminus is derivatized to —C(O)R2 where R 2 is selected from the group consisting of C1-C4 alkoxy, and —NR3R4 where R3 and R4 are independently selected from the group consisting of hydrogen and C1-C4 alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for trytophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” are defined herein as exchanges within one of the following five groups:

I. Small aliphatic, nonpolar or slightly polar residues:

Ala, Ser, Thr, Pro, Gly;

II. Polar, negatively charged residues and their amides:

Asp, Asn, Glu, Gln;

III. Polar, positively charged residues:

His, Arg, Lys;

IV. Large, aliphatic, nonpolar residues:

Met, Leu, Ile, Val, Cys

V. Large, aromatic residues:

Phe, Tyr, Trp

A “polylinker” is a nucleic acid sequence that comprises a series of three or more different restriction endonuclease recognition sequences closely spaced to one another (i.e. less than 10 nucleotides between each site).

As used herein, the term “vector” is used in reference to nucleic acid molecules that have the capability of replicating autonomously in a host cell, and optionally may be capable of transferring DNA segment(s) from one cell to another. Vectors can be used to introduce foreign DNA into host cells where it can be replicated (i.e., reproduced) in large quantities. Examples of vectors include plasmids, cosmids, lambda phage vectors, viral vectors (such as retroviral vectors).

A plasmid, as used herein, is a circular piece of DNA that has the capability of replicating autonomously in a host cell. A plasmid typically also includes one or more marker genes that are suitable for use in the identification and selection of cells transformed with the plasmid.

As used herein a “gene” refers to the nucleic acid coding sequence as well as the regulatory elements necessary for the DNA sequence to be transcribed into messenger RNA (mRNA) and then translated into a sequence of amino acids characteristic of a specific polypeptide.

A “marker” is an atom or molecule that permits the specific detection of a molecule comprising that marker in the presence of similar molecules without such a marker. Markers include, for example radioactive isotopes, antigenic determinants, nucleic acids available for hybridization, chromophors, fluorophors, chemiluminescent molecules, electrochemically detectable molecules, molecules that provide for altered fluorescence-polarization or altered light-scattering and molecules that allow for enhanced survival of an cell or organism (i.e. a selectable marker). A reporter gene is a gene that encodes for a marker.

A promoter is a DNA sequence that directs the transcription of a DNA sequence, such as the nucleic acid coding sequence of a gene. Promoters can be inducible (the rate of transcription changes in response to a specific agent), tissue specific (expressed only in some tissues), temporal specific (expressed only at certain times) or constitutive (expressed in all tissues and at a constant rate of transcription). As used herein a eukaryotic promoter is a promoter that is isolated from an organism whose DNA is localized to a nucleus bounded by a membrane. A eukaryotic promoter is not a viral promoter.

A core promoter contains essential nucleotide sequences for promoter function, including the TATA box and start of transcription. By this definition, a core promoter may or may not have detectable activity in the absence of specific sequences that enhance the activity or confer tissue specific activity.

An “enhancer” is a DNA regulatory element that can increase the efficiency of transcription, regardless of the distance or orientation of the enhancer relative to the start site of transcription.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “purified” and like terms relate to the isolation of a molecule or compound in a form that is substantially free of contaminants normally associated with the molecule or compound in a native or natural environment.

A “linker” is a molecule (or group of molecules) that serves to chemically link two disparate entities. For example a peptide linker chemically links two polypeptides via a peptide bond.

As used herein, the term “repressor” and like terms refers to the polypeptide encoded by a nucleic acid sequence comprising the sequence of SEQ ID NO: 1. In the absence of an inducer, the repressor binds to a nucleic acid operator present in a gene and inhibits transcription of the operably linked gene. Upon binding of the repressor to a specific inducer, the repressor disassociates from the operator to which it was bound thereby permitting transcription of the gene to occur.

As used herein, the term “nuclear localization signal” and like terms refers to an amino acid residue sequence that, when present in a protein, directs migration of that protein to the cell's nucleus, as evidenced by accumulation of the protein in the nucleus after biosynthesis in the cell's cytoplasm.

An operator is a nucleic acid sequence that represents the binding site for a repressor. The repressor and operator form a system for regulating a gene that is operably linked to the operator, wherein binding of the repressor to the operator inhibits transcription of the linked gene.

An inducer is a molecule, typically a low molecular weight molecule, that binds to the repressor of the present invention and causes the repressor to dissociate from an operator to which the repressor is bound.

“Operably linked” refers to a juxtaposition wherein the components are configured so as to perform their usual function. Thus, promoters operably linked to a coding sequence are capable of effecting the expression of the coding sequence; and an operator that is operably linked to a promoter (or other gene element) is capable of inhibiting transcription from the linked promoter.

As used herein the term “gene element” is intended to encompass any portion of a gene where one or more operator elements can be inserted, wherein the operator in conjunction with its corresponding repressor will reversible inhibit expression of the linked gene. For example the operator element(s) can be inserted into an intron of a gene.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

The Invention

Technological advances have made it possible to alter the genomes of animals, such as the mouse, and add or subtract genetic material relatively easily. As a result, the mouse is widely used to model mammalian development and disease. One recognized drawback has been the inability to control the expression of the altered genome experimentally once changes have been made. A reversible gene expression system could overcome this problem by enabling a target gene to be switched on and off, and without affecting the expression of non-targeted genes.

Reversible systems adapted from mammalian regulatory elements, such as heavy metal- or hormone-responsive promoters, have the disadvantage that whatever induces the targeted gene will also induce those endogenous genes that normally respond to it. In contrast, regulatory systems based on prokaryotic elements should in principle be exquisitely specific in the context of the mammalian genome.

The present invention is directed to a system for controlling phenotype in transgenic plants and animals based on elements from the lac operon of E. coli. The system of the present invention comprises two elements, the first of which is a eukaryotic gene element that has been modified to contain lac operon elements, and the second component comprises the gene encoding the lac repressor. More particularly, the lac repressor transgene of the present invention is one that has been modified to express functional levels of the repressor protein of SEQ ID NO: 3 (or a sequence that differs from SEQ ID NO: 3 by 1-15, more preferably 1-3 conservative amino acid substitutions) in a transgenic plant or animal.

In accordance with one embodiment, a lacI recombinant gene is provided that can be expressed in a mammalian cell at levels sufficient to regulate the expression of a second recombinant gene that has been modified to contain at least one copy of the lac operator. Preferably, the lacI coding sequence has the sequence of SEQ ID NO: 1, wherein the DNA sequence of the native lac repressor is altered to enable expression in a transgenic plant or animal. More particularly, the native bacterial sequence is modified in part to resemble the mammalian preferred codon usage while maintaining the same encoded amino acid sequence.

It is anticipated that minor alterations to the DNA sequence of SEQ ID NO: 1, can be made that will retain the genes ability to express a functional repressor in a transgenic plant or animal. Accordingly a nucleic acid gene construct comprising the sequence of SEQ ID NO: 1 or sequences that differ from SEQ ID NO: 1 by 1 to 100, or 1 to 50, more preferably 1 to 25 nucleotide alterations that still encode a functional repressor protein are within the scope of the present invention. These nucleotide alterations may include nucleotide deletions, insertions or substitutions of one nucleotide for another. Typically the nucleotide alteration is a simple transition from a purine to a pyrimidine or vice versa. In one embodiment a nucleic acid sequence is provided comprising the sequence of SEQ ID NO: 1 or sequences that differ from SEQ ID NO: 1 by 1 to 20, more preferably 1 to 5 nucleotide alterations, that do not alter the amino acid sequence of the encoded repressor protein. The present invention also encompasses nucleic acid sequences that hybridize (under conditions defined herein) to all or a portion of the nucleotide sequence represented by SEQ ID NO:1 or its complement and encode a repressor protein that is functional in a transgenic plant or animal.

Nucleic acid duplex or hybrid stability is expressed as the melting temperature or Tm, which is the temperature at which a nucleic acid duplex dissociates into its component single stranded DNAs. This melting temperature is used to define the required stringency conditions. Typically a 1% mismatch results in a 1° C. decrease in the Tm, and the temperature of the final wash in the hybridization reaction is reduced accordingly (for example, if two sequences having >95% identity, the final wash temperature is decreased from the Tm by 5° C.). In practice, the change in Tm can be between 0.5° C. and 1.5° C. per 1% mismatch.

The present invention is directed to the nucleic acid sequence of SEQ ID NO: 1 and nucleic acid sequences that hybridize to that sequence (or fragments thereof) under stringent or highly stringent conditions. In one embodiment the invention is directed to a purified nucleic acid sequence that encodes a functional repressor polypeptide (i.e. one capable of specific and reversible binding to its coresponding operator) that hybridizes to SEQ ID NO: 1 or its complement under highly stringent or stringent conditions. In accordance with the present invention highly stringent conditions are defined as conducting the hybridization and wash conditions at no lower than −5° C. Tm. Stringent conditions are defined as involve hybridizing at 68° C. in 5×SSC/5×Denhardt's solution/1.0% SDS, and washing in 0.2×SSC/0.1% SDS at 68° C. Moderately stringent conditions include hybridizing at 68° C. in 5×SSC/5×Denhardt's solution/1.0% SDS and washing in 3×SSC/0.1% SDS at 42° C. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

As reported in the literature, simply modifying the bacterial sequence to resemble the preferred mammalian codon usage fails to produce a functional product in eukaryotic cells. Such a modified repressor gene (the “synlacI” gene construct, described in Scrable and Stambrook, Genetics 147, 297-304 (1997)), produced high levels of mRNA, but no functional protein. Northern blot analysis of total RNA hybridized to a lac repressor probe identified a single transcript in RNA from animals transgenic for the bacterial repressor construct, but a doublet was detected in RNA from animals transgenic for the synlacI gene construct.

Through the use of a series of chimeric repressor gene constructs made by exchanging the 5′ region of the wild type bacterial repressor with the corresponding region of the synlacI gene construct it was determined that the mRNA transcribed from the synlacI sequence was being improperly processed. More particularly, the chimeric constructs revealed that the synlacI mRNA is improperly spliced as a result of a sequence present in the first 36 bp of the synlacI coding region. Within these first 36 bp of coding sequence, the wild type bacterial repressor and the synlacI gene constructs differ only by four bases, and the changes are in all cases simple transitions from a purine to a pyrimidine or vice versa. In particular, the transitions made in the original synlacI coding region comprise A to G at position 15, G to A at position 18, T to C at position 21 and T to C at position 27.

The synlacI repressor gene construct was modified to incorporate the amino acid transitions at positions 15, 18, 21 and 27 (thus reverting the sequence back to the wild type bacterial sequence) however further investigation revealed that in addition to splicing, there was a second problem with the synlacI coding region that affected the expression of functional repressor. In particular, using a series of chimeric repressor gene constructs made by exchanging the 3′ region of the wild type bacterial repressor with the corresponding region of the synlacI gene construct it was determined that functional lac repressor activity was being blocked by the region of the synlacI sequence in the dimerization domain. Accordingly, the coding sequences between the EcoRV and the PvuII restrictions sites of the synlacI construct were replaced with wild type bacterial lacI coding sequences. This repressor encoding sequence, represented as SEQ ID NO: 1 is correctly spliced and translated in transfected eukaryotic cells.

A gene construct comprising the nucleic acid sequence of SEQ ID NO: 1 operably linked to the β-actin promoter (construct 3′C4) was used to create transgenic mice, but surprisingly this construct expressed the repressor only in the testis, resembling the expression pattern of a transgene composed entirely of bacterial coding sequence. The content of CpG dinucleotides in the coding sequence is a major determinant of transcription in animals. In the nucleic acid construct of SEQ ID NO:1, the replacement of the synlacI sequence between the EcoRV and PvuII sites with the corresponding nucleic acid sequence from the bacterial lacI changed the 3′ terminal region from one devoid of CpG (and ubiquitously expressed) to one that is CpG rich (and expressed only in the testis). However, altering the sequence to remove the CpG rich region leads to a gene that is transcribed but not translated as noted above.

In an attempt to understand this problem, CpG-density maps were prepared for each repressor construct and aligned with the CpG-density map of the β-actin gene. This analysis revealed two segments downstream from the actin promoter that are free of CpG dinucleotides. In repressor constructs that contained CpG dinucleotides in a corresponding region, the expression of the repressor was limited to the testis. Thus to overcome this problem the repressor coding region was flanked with non-coding regions to move the repressor coding region farther away from the promoter used to express the repressor coding region. Structural repositioning of the 3′ CpGs (construct R) resulted ubiquitous expression of the repressor product in transgenic animals.

Therefore, the expression of lacI from the β-actin promoter in transgenic animals depends on the density and position of CpG-rich regions in the lacI transgene. In summary, the overall gene construct should be prepared so that at least two small regions (of about 100 bp in length) that lie approximately 600 and 800 bp downstream of the transcription start site are devoid of CpG dinucleotides. In accordance with one embodiment a repressor gene construct is prepared comprising a eukaryotic promoter operably linked to a eukaryotic intron that is in turn operably linked to the lacI coding sequence, wherein the lacI coding sequence is operably linked to the 3′ untranslated region of a eukaryotic gene. The inclusion of the eukaryotic intron sequences is also believed to be important in optimizing the transport of the mRNA from the nucleus to the cytoplasm and subsequent translation of the repressor protein. The intron sequence used is not critical provided that it has the necessary spice junctions to be properly excised from the encoded mRNA. Furthermore, in one embodiment (when using the β-actin promoter, for example) the intron provides adequate spacing so that two 100 bp regions devoid of CpG dinucleotides are located approximately 600 and 800 bp downstream of the transcription start site. In one preferred embodiment the eukaryotic promoter used to drive the expression of the repressor is a mammalian promoter, and the intron and the 3′ untranslated region of the modified repressor gene are selected from β-globin, and more particularly the gene construct comprises the sequence of SEQ ID NO: 4.

One goal of the present invention is to use the repressor gene of the present invention to regulate the expression of other genes in vivo. Therefore, to obtain such regulation of eukaryotic genes it is necessary to have the expressed repressor transported into the cell's nucleus. In accordance with one embodiment the repressor encoding nucleic acid sequence of the present invention is operably linked to a nuclear localization signal sequence (NLS). Nucleus-targeting sequences have been described for a variety of proteins and typically are short amino acid residue sequences of about 5-15 residues. Any of the NLS sequences that have been previously described in the literature are suitable for use in accordance with the present invention and include those described by Jans et al., BioEssays, 22:532-544 (2000), the disclosure of which is incorporated herein.

In accordance with one embodiment the SV40 nuclear localization signal (NLS) is used to direct the recombinant repressor protein to the nucleus. The SV40 Large T antigen has been reported to contain a seven amino acid residue sequence (ProLysLysLysArgLysVal; SEQ ID No. 7) that defines a minimum region of the Large T antigen required for nuclear targeting (see Kalderon et al., Cell, 39:499-509 (1984)). The SV40-derived nuclear location signal has been engineered into several different proteins to cause them to accumulate in the nucleus of a cell, including bacteriophage T7 RNA polymerase into mammalian cell nuclei (Dunn et al., Gene, 68:259-266, 1988), and into yeast cell nuclei (Benton et al., Mol. Cell. Biol., 10:353-360, 1990). In accordance with one embodiment the nuclear localization signal of SV40 is linked to the repressor encoding squence of SEQ ID NO: 1 to produce the nucleic acid sequence of SEQ ID NO: 2.

Although the SV40 nuclear location sequence is used in one embodiment of the present invention, other nuclear location sequences can be utilized. For example, the NLS of the adenovirus E1 a gene product (LysArgProArgPro; SEQ ID NO: 8) that is located at the extreme carboxyl terminus of E1a (see Lyons et al., Mol. Cell. Biol., 7:2451-2456 (1987)) can be utilized. In addition, other NLS sequences have been identified in both higher eukaryotes and in the yeast, Saccharomyces cerevisiae and are suitable for use in accordance with the present invention. See, for example, the review by Silver et al., in Protein Transfer and Organelle Biogenesis”, Das et al., eds., Academic Press, Inc., N.Y., P. 747-769 (1988). Furthermore, assays for identifying proteins and protein regions having a nucleus-targeting sequence have been described. See, for example Parnaik et al., Mol. Cell. Biol., 10:1287-1292 (1990).

The location of a nucleus-targeting sequence relative to the sequence encoding the recombinant repressor of this invention can vary, so long as the resultant protein exhibits the requisite properties. The NLS sequence is preferably located either at the amino or the carboxy terminus of the encoded repressor protein. In accordance with one embodiment the amino terminal location of a nucleus-targeting sequence is within about 5 amino acid residues of the amino terminus of the inducible lac repressor. Particularly preferred is a construct where the nucleus-targeting sequence begins as the second amino acid residue after the amino-terminal methionine encoded by the initiation codon (ATG). In the alternative embodiment wherein the NLS is located at the carboxy terminus of the repressor, the NLS coding sequence is located within 100 bases upstream, and more preferably 1-3 bases upstream, from the termination codon of the DNA segment that codes for the inducible lac repressor. In one embodiment the NLS sequence is linked to the repressor coding sequence through the use of a short nucleotide linker.

Although fusions of the NLS to the repressor at either the extreme 5′ or 3′ end could be made that did not adversely affect repression or induction, an ideal configuration utilizes a linker between lacI and the SV40 NLS. In one embodiment the linker comprises a three amino acid linker (Ser-Ser-Leu coded for by AGC-AGC-CTG) between the end of lacI and the SV40 NLS. Accordingly, in one preferred embodiment the NLS coding sequence is operably linked by the AGC-AGC-CTG spacer oligonucleotide to the 5′ terminal codon prior to a termination codon to generate the sequence of SEQ ID NO: 2.

In accordance with one embodiment, a mammalian “gene” was assembled from the modified lac repressor sequence with an NLS and the full-length human beta-actin promoter fused to the intron of a genomic fragment of the rabbit beta-globin gene. The lad coding sequence was cloned in the remainder of the beta-globin fragment, which included the 3′UTR and polyadenylation signal sequence. The sequence consisting of rabbit β-globin intron 2, the lacI coding sequence and the remainder of the beta-globin fragment including the 3′UTR is provided as SEQ ID NO: 4. The sequence consisting of rabbit β-globin intron 2, the lacI coding sequence, the remainder of the beta-globin fragment including the 3′UTR and the polyA signal sequence from SV40 is provided as SEQ ID NO: 11. More particularly, in one embodiment the modified lac repressor gene construct comprises:

1) The 4.3 kb promoter region from the human β-actin promoter from the EcoRI site up to the AscI site 70 bp upstream of the start of translation (the reverse complement of base pairs 3,483,536 to 3,479,221 of NT_{—}007844, number refers to the Genbank accession number).

2) rabbit β-globin intron 2 from the blunted NcoI site through the EcoRI site in exon 3. (bases 31528-32032 of m18818)

3) The lacI coding region was inserted at the EcoRI site:

The first 27 bp are: atg aaa cca gta acg tta tac gat gtc (SEQ ID NO: 9). It then continues as the synlacI sequence given in (Scrable and Stambrook, 1997) until the EcoRV site at +800 of the coding sequence. There are then 150 bp identical to the wtlacI sequence, up to the PvuII site at +950 of the coding sequence (bases 881-1030 of j01637). There are then 129 bp identical to the synlacI sequence (up to the NLS indicated in (Scrable and Stambrook, 1997)). A linker region and SV40 large T-Antigen NLS are attached to the 3′ end of the coding region: agc agc ctg agg cct ccc aag aag aag cga aag gtg tga (SEQ ID NO: 10)

4) The rabbit α-globin fragment continues downstream of the lacI coding region to include the rest of exon 3 and the 3′ UTR with a polyadenylation signal sequence (from the EcoRI site to the PvuII site; the reverse complement of bases 32033-32571 of m18818). It is followed by the polyA signal sequence from SV40 (from the HpaI site to the BamHI site; bases 2669-2539 of j02400). Following the SV40 sequence is a 276 bp fragment of the cloning vector pBR327 (SEQ ID NO:12; from the BamHI site a bp 375 to the SalI site at bp 651). All of the β-globin sequences, the SV40 polyA signal sequence, and the vector fragment are as described in (Katsuki et al., 1988). This gene construct can be introduced into eukaryotic cells, and more particularly the construct can be used to prepare transgenic animals, such as mice and other mammals, containing such a construct.

The eukaryotic promoter used to express the repressor gene sequence can be selected from any of the known eukaryotic promoters, including promoters that are constitutive, temporally regulated or are tissue specific. The use of tissue- or cell type-specific promoters in conjunction with the modified lac repressor of the present invention will confer regional specificity on repressor expression and function. In one embodiment the promoter is a mammalian promoter, and more particularly a constitutive mammalian promoter.

In another embodiment of the present invention, nucleic acid sequences encoding the modified lac repressor protein can be inserted into expression vectors and used to transfect cells to express the repressor protein in the target cells or to generate additional copies of the construct. In accordance with one embodiment, the nucleic acid sequence of SEQ ID NO: 1, SEQ ID NO: 2 or SEQ ID NO: 4 are inserted into an expression vector in a manner that operably links the gene sequences to the appropriate regulatory sequences, and the recombinant repressor is expressed in a host cell. Suitable host cells and vectors are known to those skilled in the art.

The expression vectors contemplated by the present invention are at least capable of directing the replication, and preferably also expression, of a structural gene operatively linked to the vector. In one embodiment, a vector contemplated by the present invention includes a procaryotic replicon, i.e., a DNA sequence having the ability to direct autonomous replication and maintenance of the recombinant DNA molecule extrachromosomally in a procaryotic host cell, such as a bacterial host cell. Such replicons are well known in the art and include OriC. In addition, those embodiments that include a procaryotic replicon may also include a gene whose expression confers a selective advantage such as amino acid nutrient dependency or drug resistance to the transformed bacterial host cell that allows selection of transformed clones. Typical bacterial drug resistance genes are those that confer resistance to antibiotics such as ampicillin, tetracycline, kanamycin, and the like.

Expression vectors compatible with eukaryotic cells, preferably those compatible with cells of vertebrate or mammalian species, can also be used to form the recombinant DNA molecules of the present invention. Eukaryotic cell expression vectors are well known in the art and are available from several commercial sources. Typically, such vectors are provided containing convenient restriction sites (i.e. a polylinker) for insertion of the desired gene. Typical of such vectors are pSVL and pKSV-10 (Pharmacia), pBPV-1/pML2d (International Biotechnologies, Inc.), and pTDT1 (ATCC, #31255).

In preferred embodiments, the eukaryotic cell expression vectors used to construct the recombinant DNA molecules of the present invention include a selectable phenotypic marker that is effective in a eukaryotic cell, such as a drug resistance selection marker or selective marker based on nutrient dependency. For example, drug resistance markers suitable for use in the present invention include the the neomycin phosphotransferase (neo) gene. (Southern et al., J. Mol. Appl. Genet., 1:327-341, 1982), and the hygromycin resistance gene.

Nucleic acid sequences encoding the recombinant repressor protein may be introduced into a cell or cells in vitro or in vivo using standard techniques, including the use of liposomes, viral based vectors, electroporation or microinjection. Accordingly, one aspect of the present invention is directed to transgenic cell lines that comprise the nucleotide sequence of SEQ ID NO: 1, SEQ ID NO: 2 or SEQ ID NO: 4. In one embodiment a transgenic cell is provided that comprises the nucleic acid sequence of SEQ ID NO: 4.

The present invention also encompasses gene constructs that are regulated by the recombinant repressor of the present invention. These gene constructs comprise an operator operably linked to a gene. In one preferred embodiment the operator is operably linked to eukaryotic promoter, wherein the promoter is operably linked to an open reading frame. An operator is “operably linked” to a gene/promoter when transcription of the gene is inhibited in the presence of the repressor and absence of the inducer, and the inhibition is reversed when the inducer of the repressor is also present. In one preferred embodiment the eukaryotic promoter is a mammalian promoter.

The lac operator is a short (˜20 bp) DNA sequence that can be synthesized with flanking ends to allow it to be inserted into an available restriction site, or used with the polymerase chain reaction to replace a segment of DNA to convert a mammalian promoter into a regulatable version. This has been done for the murine H-2K^{b} promoter, the human serine tRNA promoter, and the murine PGK promoter, as previously reported.

The present invention also encompasses nucleic acid constructs wherein an operator is operably linked to a eukaryotic promoter and the promoter is operably linked to a polylinker. This nucleic acid construct can be utilized to conveniently insert the coding region of gene so that the transcription of the gene can be regulated by a repressor protein interacting with the operator. In one preferred embodiment the eukaryotic promoter is a mammalian promoter.

Operators function to control the expression of a gene by a variety of mechanisms. The operator can be positioned within a promoter such that the binding of the repressor covers the promoter's binding site for RNA polymerase, thereby precluding access of the RNA polymerase to the promoter binding site. Alternatively, the operator can be positioned downstream from the promoter binding site, thereby blocking the movement of RNA polymerase down through the transcriptional unit. In particular, it has been demonstrated, first in E. coli and later in rabbit kidney cells (Deuschle et al., Science, 248(4954), 480-483, (1990)) expressing lac repressor, that a single operator inserted into the middle of a transcription unit could interrupt polymerase and cause premature termination of nascent RNA molecules. Thus it is anticipated that operators inserted into introns (or other gene elements) of mammalian genes will function as transcription terminators, which would alter the mechanism but not the outcome of gene regulation by lac in mammalian cells and animals.

Multiple operators can be inserted into a gene construct to bind more than one repressor. The advantage of multiple operators is several fold. First, tighter blockage of RNA polymerase binding or translocation down the gene can be effected. Second, when spaced apart by at least about 70 nucleotides and typically no more than about 1000 nucleotides, and preferably spaced by about 200 to 500 nucleotides, a loop can be formed in the nucleic acid by the interaction between a repressor protein bound to the two operator sites. The loop structure formed provides strong inhibition of RNA polymerase interaction with the promoter, if the promoter is present in the loop, and provides inhibition of translocation of RNA polymerase down the transcriptional unit if the loop is located downstream from the promoter.

There are two considerations that should be taken into account when modifying a eukaryotic promoter for use in a transgenic animal: where to position the operators in the promoter, and selecting the operator sequence to use. Operator position is important not only to achieve optimum repression, but also to minimize the effect of promoter modification on basal promoter activity. Operator sequence is important to ensure that induction of promoter activity is as successful as repression. Typically the promoter will be modified to include an operator a few base pairs upstream of a transcription start site and a second operator identical in sequence to the first operator approximately 93 base pairs downstream from the first operator. In one embodiment the operators have the sequence of SEQ ID NO: 6 and the first operator is located approximately 1-3 base pairs upstream of the transcription start site.

In E. coli, the transcription start site (tss) of the regulatable lac promoter is flanked by the primary operator just downstream of the start site (O₁) and a secondary operator O₂, located 93 bp upstream. Selective pressure over eons of time appears to have positioned these two operators in a nearly perfect physical relationship to each other, as an optimum distance for repression has been found experimentally to be 92.5 bp. (Interestingly, maximal repression was also obtained experimentally at an operator spacing of 70.5 bp and at 115.5, the natural operator spacing in the gal operon). Experiments in E. coli have also demonstrated that repression by O₁ at its natural position increases up to 50-fold in the presence of an optimally positioned auxiliary operator, which can be attributed to stable DNA loop formation. A third operator (O_{z}) lies within the coding sequence of the beta-galactosidase gene 401 bp downstream of O₁.

In preparing the tyrosinase promoter for regulation (see Example 1), much of the original architecture of the bacterial promoter was replicated. Operators were inserted 9 bp downstream (O₁) and 176 bp upstream (O₂) of the tss so that they flanked the tss in the promoter. The addition of the beta-galactosidase gene downstream brought the auxiliary operator in the lacZ coding sequence (O_{z}) into a position relative to the operators in the promoter that mimicked the spacing of that in the lac operon. This configuration was tested in vitro and found to give tight regulation when co-transfected with lacI DNA and grown in the presence or absence of IPTG.

The auxiliary operator in lacZ was eliminated by replacing the beta-galatosidase gene with the gene encoding the A-chain of diphtheria toxin, and a new third operator was inserted 500 bp upstream of O₂. Stringency of regulation was assayed by counting the number of dead cells that resulted from induction of the toxin by IPTG. Moving the third operator from a position downstream to a position upstream did not appear to attenuate repression, as there was the same low number of dead cells in untransfected cells as in cells co-transfected with the toxin gene and the lac repressor. Thus, the third operator may function in the context of a regulatable mammalian promoter in much the same way it does in the bacterial operon, where it serves to sequester excess repressor molecules in close proximity to sites where they are actively being used.

In an attempt to avoid having to flank the tss with operators, primary and secondary operators were both placed downstream of tss to generate a regulatable version of the murine p53 promoter. In conjunction with the operator in lacZ, this resulted in about 90% repression in co-transfection assays with lacI and IPTG. Attempts to simplify the modifications even further by using only one upstream operator (O₁, or O₂) with O_{z}, or with tandem repeats of two or three primary operators with O_{z}. were less successful. These manipulations with the p53 promoter show that it is possible to achieve repression without having to flank the tss with operators if one is willing to sacrifice some degree of control. This may be a viable compromise in situations when total repression of gene expression is not required.

Another solution to flanking operators would be to place operators in positions upstream of tss only. One of the most tightly lacO-regulated promoters known is the modified SV40 immediate early promoter constructed by Figge et al. A single operator was inserted between tss and the TATA box, creating a new tss the same distance from TATA as the original tss had been. This single operator confers virtually complete repression on the promoter in the presence of lacI. When lacZ was replaced by CAT (which simultaneously eliminated O_{z}), the same level of repression was obtained. This strategy was also used to modify the H-2K^{b} promoter from the MHC locus of the mouse. A single operator positioned between tss and the TATA box conferred regulation on 80% of mouse L-cell clones stably transfected with lacI and an H-2K^{b} lacZ reporter gene. At least two of the clones exhibiting tightly regulated β-galactosidase expression contained only a single copy of lacI. Finally, as noted above a single operator inserted into the middle of a transcription unit could interrupt polymerase and cause premature termination of nascent RNA molecules.

Any of the operator sequences known to those skilled in the art are suitable for use in the present invention. There are two sequence variants of the lac operator that have been used in experimental systems. The first, referred to as the wild-type sequence, is the sequence found at the primary operator site (O₁) in the regulatable promoter of the bacterial operon. The sequence is an imperfect palindrome whose mirror image reflects about a central unpaired guanine. The second operator sequence is an “ideal” version of the first in which mismatched bases have been replaced to create a perfect palindome, and the central unmatched base has been removed. No obvious significant difference in the efficacy of these two operator sequences has been detected. The wildtype-type sequence, with its mismatches, is less likely to self-anneal and for that reason may be easier to handle in the lab.

In accordance with one embodiment two optimized operators derived from the lac operon and having the sequence ATTGTGAGCGCTCACAAT (SEQ ID NO: 6) or TGTGGAATTGTGAGCGCTCACAATTCCACA (SEQ ID NO: 5) are use in accordance with the present invention. A comparison of these two operators has been conducted in mammalian cells. Each of these two operators were inserted into the Pol III promoter of a human serine amber suppressor tRNA (Su⁺ tRNA) gene at the −1 position. Suppressor activity in mammalian cells was measured as a function of the ability of Su⁺ tRNA to suppress the UAG nonsense codon in a CAT reporter gene co-transfected with lacI. With the 18 bp operator it was possible to reduce suppressor activity by 75-85%, but with the 30 bp operator activity was reduced by 98%.

The present invention also encompasses a pack or kit comprising two gene constructs for preparing transgenic mammals for in vivo regulation of gene expression. The first construct comprises a eukaryotic promoter linked to the modified repressor gene of the present invention. In one embodiment, the first construct comprises an intron region linked to the lacI coding sequence of SEQ ID NO: 2, which is in turn linked to the 3′ untranslated region of a eukaryotic gene. The intron is operably linked to the lacI coding sequence, and thus is properly excised from the mRNA prior to translation of the mRNA. In one preferred embodiment the first construct comprises the sequence of SEQ ID NO: 4 operably linked to a eukaryotic promoter. The second gene construct comprises a eukaryotic promoter that has been modified to incorporate one or more lac operators. Furthermore, the modified eukaryotic promoter of the second construct is operably linked to either the coding sequence of a protein or to a polylinker (i.e. a nucleic acid region containing multiple restriction endonucleases in close proximity). In accordance with one embodiment the eukaryotic promoters of the two constructs are mammalian promoters.

The two constructs of the kit can be packaged in a variety of containers, e.g., vials, tubes, microtiter well plates, bottles, and the like. Other reagents can be included in separate containers and provided with the kit; e.g., positive control samples, negative control samples, buffers, cell culture media, etc. Preferably, the kits will also include instructions for use.

One aspect of the present invention is directed to non-human transgenic animals that comprise the nucleic acid constructs of the present invention. A transgenic plant or animal in accordance with the present invention has at least 1 cell containing a gene construct of the present invention. In preferred embodiments all the cells of the transgenic plant or animal comprise one or more transgenes of the present invention inserted into the cell's genome. A transgene is a DNA sequence integrated at a locus of a genome, wherein the transgenic DNA sequence is not otherwise normally found at that locus in that genome. Transgenes may be made up of heterologous DNA sequences (sequences normally found in the genome of other species) or homologous DNA sequences (sequences derived from the genome of the same species). The transgenic organisms encompassed by the present invention include any of the multicellular eukaryotic organisms that undergo sexual reproduction by union of gamete cells. Preferred organisms include mammals, birds, fish (i.e. zebrafish), amphibians (i.e. frogs), and plants, including both gymnosperms and angiosperms. In one preferred embodiment the transgenic animal is a non-human mammal, including but not limited to sheep, cows, pigs, horses, rabbits, primates and rodents, such as mice or rats, and the like.

One embodiment of the present invention is directed to transgenic mice that comprise a nucleic acid sequence comprising a mammalian promoter operably linked to rabbit β-globin intron 2, which is operably linked to the lacI coding region, which is linked to the 3′ untranslated region of the rabbit β-globin gene. This construct allows for the expression of lac repressor in amounts sufficient to inhibit gene constructs that contain one or more copies of the lac operator in the 5′ end of the gene (i.e. near the transcriptional start site of the gene).

In one embodiment a non-human transgenic mammal is provided wherein the cells of the mammal comprise a repressor transgene that is stably integrated in its genome. The repressor transgene comprises the nucleic acid sequence of SEQ ID NO: 4 operably linked to a eukaryotic promoter. In another embodiment a non-human transgenic mammal is provided wherein the cells of the mammal comprise an operator (capable of interacting with the repressor encoded by SEQ ID NO: 4) operably linked to a promoter (or some other gene element), wherein the promoter is operably linked to a sequence that encodes a protein. In one preferred embodiment the non-human transgenic mammal's cells comprise both the repressor transgene as well as a second gene that comprises a eukaryotic promoter, modified to incorporate one or more lac operators, operably linked to the coding sequence of a protein. Thus the expression of the second recombinant gene construct can be regulated by administering lactose or a lactose analog, such as IPTG, to the mouse. Transgenic animals that comprise both gene constructs can be prepared by crossing the two respective transgenic mammals to produce a progeny transgenic mammal containing the transgene of each parent transgenic mammal. The procedure generally involves mating male and female transgenic mammals (founders) to produce offspring, at least some of which will be transgenic mammals containing the transgenes of both parents, i.e., a hybrid transgenic mammal.

The transgenic animals of the present invention can be produced using methods well known in the art. See for example, Wagner et al., U.S. Pat. No. 4,873,191 (Oct. 10, 1989); Hogan et al., Manipulating the Mouse Embryo: A Laboratory Manual, Cold Springs Harbor, N.Y. (1987); Capecchi, Science, 244:288-292 (1989); and Luskin et al., Neuron 1:635-647 (1988). One technique for transgenically altering a mammal is to microinject a gene construct into the male pronucleus of the fertilized mammalian egg to cause one or more copies of the gene construct to be retained in the cells of the developing mammal. The gene construct is isolated in a linear form with most of the sequences used for replication in a host cell removed. Linearization and removal of excess vector sequences results in a greater efficiency in production of transgenic mammals. See for example, Brinster, et al., Proc. Natl. Acad. Sci., USA, 82:4438-4442 (1985). Usually up to 40 percent of the mammals developing from the injected eggs contain at least 1 copy of the recombinant DNA in their tissues. These transgenic mammals usually transmit the gene through the germ line to the next generation. The progeny of the transgenically manipulated embryos may be tested for the presence of the construct by Southern blot analysis of a segment of tissue. For example, a small part of the tail of the animal is used for this purpose. The stable integration of the rDNA into the genome of the transgenic embryos allows permanent transgenic mammal lines carrying the rDNA to be established. An exemplary preparation of a transgenic mouse is provided in the Examples.

Alternative methods for producing a non-human mammal containing one of the gene constructs of the present invention include infection of fertilized eggs, embryo-derived stem cells, totipotent embryonal carcinoma (Ec) cells, or early cleavage embryos with viral expression vectors containing the gene construct. See for example, Palmiter et al., Ann. Fev. Genet., 20:465-499 (1986) and Capecchi, Science, 244:1288-1292 (1989). The infection of cells within an animal using a replication incompetent retroviral vector has been described by Luskin et al., Neuron, 1:635-647 (1988). The frequency of obtaining transgenic animals by retroviral infection of embryos can be as high as that obtained by microinjection of the rDNA and appears to depend greatly on the titre of virus used. See, for example, van der Putten et al., Proc. Natl. Acad. Sci., USA, 82:6148-6152 (1985).

Another method of transferring new genetic information into the mouse embryo involves the introduction of the gene construct into embryonic stem cells (usually by electroporation) and then introducing the embryonic stem cells into the embryo. The embryonic stem cells can be derived from normal blastocysts and these cells have been shown to colonize the germ line regularly and the somatic tissues when introduced into the embryo. See, for example, Bradley et al., Nature, 309:255-256 (1984).

A transgene containing an operator sequence can be regulated in accordance with the present invention in a transgenic animal by supplying or removing the inducing agent. For example, to induce the expression of a transgene that is suppressed by the operator/repressor system of the present invention, a cell within the organism that contains the relevant trangene is contacted with an effective amount of inducer for a time period sufficient for the inducer to be taken up by the cell and for the inducer to bind the repressor. The repressor dissociates from the operator, and the gene is expressed within that cell.

An effective amount of inducer is an amount sufficient to bind repressor and derepress the operator-regulated reporter gene, thereby causing expression of the reporter gene product in the contacted cell. Preferred amounts of inducer effective to bind repressor and derepress the regulated gene depend on degree and extent of derepression desired. Typically, an effective amount of inducer to be contacted with a cell to be regulated is in the range of 10 picomolar (pM) to 500 millimolar (mM), preferably about 1 mM to 200 mM, and more preferably about 50 mM. Thus in one embodiment, to induce a transgene in a transgenic animal, an inducer is administered to the animal in an amount sufficient to produce a blood concentration having an effective amount of inducer.

The inducer can be administered to the transgenic animal by a variety of means to deliver the inducer to the cell (i.e., contact the cell) containing the eukaryotic gene regulation system to be induced, and depends in part on the cell type to be induced and tissue in which the cell is located in the organism. Administration can be topical, oral, as by ingestion, intravenous, intramuscular, intradermal or intraperitoneal, and can be accomplished by a single dose, by repeated doses, or by continuous infusion. In one embodiment, continuous infusion is obtained through the use of an implantable osmotic pump. One preferred route of adminstration is orally, including for example, placing the inducer in the animal's food or water. Repression of the gene product can be reestablished simply by ceasing the adminstration of the inducing agent.

The inducer used in the present invention is a molecule, typically a low molecular weight molecule, that binds to the lac repressor polypeptide of the present invention and causes the repressor to dissociate from a nucleic acid operator sequence to which it is bound. More particularly, the lac repressor is induced by a class of galactoside derivatives that are exemplary of inducers for the present invention. See, for example Miller, J. H., in “The Operon”, p. 31-88, 34, Miller et al., eds., Cold Spring Harbor Laboratory, New York, 1980; and Jacob et al., J. Mol. Biol., 3:318-356, 324 (1961).

In one embodiment, lac repressor inducers of the present invention are derivatives of galactoside that are modified to increase the half-life of the derivative in physiological solutions. Preferred modified galactosides are thiogalactoside derivatives such as the prototype isopropyl-beta-D-thiogalactoside (IPTG). Modified thiogalactosides that are selectively taken up in specific tissues of all animal are described in U.S. Pat. No. 5,589,392, the disclosure of which is incorporated herein. In particular, by careful selection of a modified thiogalactosides, one can direct the uptake, and therefore the induction, to specific tissues or cell types based on the properties of the modifiedthiogalactoside.

As described in detail in the Examples the present regulation system can be used to control the expression of a gene in a transgenic animal. The lac repressor was demonstrated to effectively regulate pigmentation in the mouse by controlling the activity of the murine tyrosinase promoter into which lac operators were inserted to control the expression of a visible marker, tyrosinase. Regulation was also determined to be fully reversible. In addition to the tyrosinase promoter, the promoters of the human Huntington's disease gene locus and the murine Arc gene have also been modified to insert the functional operator sequence of SEQ ID NO: 6.

Expression of the modified genes can be switched on and off easily during embryogenesis and in the adult mouse by supplementing drinking water with a low concentration of IPTG. In accordance with one embodiment the drinking water of the transgenic animal was replaced with 10-12.5 mM IPTG in light-protected water bottles to induce expression. Expression of reporter genes can also be induced in vivo by intraperitoneal injection of IPTG. In an exhaustive study on the pharmacokinetics of potential lac inducers in mammalian cells and animals, IPTG was rapidly taken up by facilitated transport into the tissues of the animal, where it reached high levels in cells in 2-4 hours. Nuclear uptake of inducer averaged 18% of the total cell uptake, estimated to be a 1000-fold higher relative concentration of inducer molecules to repressor molecules than is required for maximal induction in E. coli. Tissue distribution in the adult animal was widespread (spleen, liver, lung, kidney, brain, and adipose tissue), and based on the results described in Example 2, IPTG can cross the placenta to induce gene expression in embryos. Tissues were found to have a large capacity for inducer uptake, but it was rapidly cleared from the blood, which allowed cells to survive the initial high doses that were used to achieve maximal uptake. The synthetic sugar was not metabolized in the animal and remained functionally active for at least 4 hours after introduction into the bloodstream. Gene expression can then be switched off by removing IPTG.

The lac operator-repressor system brings the temporal dimension of mammalian gene expression in the animal under experimental control that is both reliable and predictable, and should make it possible to introduce even lethal mutations into the mouse genome routinely and to study them at the organismal level. Elimination of the requirement for any viral elements, furthermore, should result in improved reliability, predictability, and consistency relative to other available systems. For these reasons, the system should prove to be of general utility for introducing lethal mutations and creating true phenocopies of genetic diseases in the laboratory mouse.

The regulatory control of exogenous genes in vivo or in vitro provides a wide variety of commercial and research applications. Transgenic animals containing an exogenously-added regulatable gene provide a research tool to investigate the control of eukaryotic genes, allow the preparation of animals with altered growth characteristics, allow the development of animal models for human disease gene therapy, and provides a system to study developmental genes and tumorigenesis. Inducible expression systems based on prokaryotic elements are particularly useful because they allow for precise regulation of the exogenous gene without altering the expression of the other genes present in a cell.

The lac operator-repressor system of the present invention is used in accordance with one embodiment to regulate both genes that are introduced experimentally into the resident genome and genes that are already there. In accordance with one embodiment endogenous genes are targeted for the insertion of operators to regulate the expression of the targeted endogenous gene in vivo. More particularly, the present invention encompasses transgenic animals that comprise an endogenous gene having one or more operators inserted into a gene element of the gene. The operator sequences can be inserted into the endogenous genes using any of the standard techniques for introducing gene constructs and inserting the genes into the genome of the cell. In one embodiment the introduced operator constructs are flanked with sequences homologous to the endogenous gene and the operator is inserted into the gene through the use of homologous recombination. The operator sequences can be inserted at any non-coding site of the gene including the promoter, introns and 5′ and 3′ untranslated regions of the gene, with one preferred site being the intron regions.

In one embodiment a method of regulating the expression of a gene in a transgenic animal is provided. The method comprises providing a transgenic animal wherein the cells of said animal comprise a first nucleic acid sequence comprising the sequence of SEQ ID NO: 4, and a second nucleic acid sequence comprising an operator operably linked to said gene, and contacting the cells of the transgenic animal in vivo with an inducer of the repressor. In one embodiment the transgenic animal is created by first introducing and inserting into the genome of the animal a DNA construct comprising an operator sequence. In one embodiment the introduced operator sequence is inserted into an endogenous gene to operably link the operator to the endogenous gene. In an alternative embodiment the introduced DNA construct further comprises a gene that is operably linked to the operator and the gene is inserted into the genome.

In accordance with one embodiment of the present invention an operator targeting vector is provided that is designed for inserting operators into the introns of endogenous genes. The vector construct comprises an operator and a reporter gene construct, wherein the reporter gene construct is flanked by direct repeats of a site-specific recombinase site. Stating that the reporter construct is flanked means that the target sites may be directly contiguous with the reporter gene or there may be one or more intervening sequences present between one or both ends of the reporter gene and the target sites. The reporter gene construct further comprises a consensus 3′ spice site upstream of the reported gene. In one embodiment the operator targeting construct comprises one or more OCR element that comprise two lac operator sequences separated by 150 or 200 bp of spacer nucleotides. In constructs containing multiple OCR elements, the elements are each separated by 400 bp of spacer nucleotides. The sequence of the spacer nucleotides is not critical, provided that it gives the desired spacing. The construct can also include sequences homologous to the target endogenous gene to allow for homologous recombination.

The site-specific recombinase sites and the corresponding site-specific recombinase used in the present invention may include any enzyme system wherein the enzyme is capable of being functionally expressed in eukaryotic cells, and catalyzes conservative site-specific recombination between its corresponding target sites. For reviews of site-specific recombinases, see Sauer (1994) Current Opinion in Biotechnology 5:521-527; Sadowski (1993) FASEB 7:760-767; the contents of which are incorporated herein by reference. Methods of using site-specific recombination systems to excise DNA fragments from chromosomal or extrachromosomal plant DNA are known to those skilled in the art. The bacteriophage P1 loxP-Cre and the Saccharomyces plasmid FRT/FLP site-specific recombinations systems have been extensively studied. For example, Russell et al. (1992, Mol. Gen. Genet. 234:49-59) describe the excision of selectable markers from tobacco and Arabidopsis genomes using the loxP-Cre site-specific recombination system.

The reported gene can be any gene sequence that encodes a detectable marker. Preferred markers include selectable markers (such as antibiotic resistant genes) and fluorescent markers. A 3′ acceptor splice sequence is provided upstream of the reported gene. Consensus splice sequences are well know to those skilled in the art and include those described in U.S. Pat. No: 5,744,326, the disclosure of which is incorporated herein. In one embodiment the 3′ acceptor splice site comprises a series of pyrimidines followed by AG.

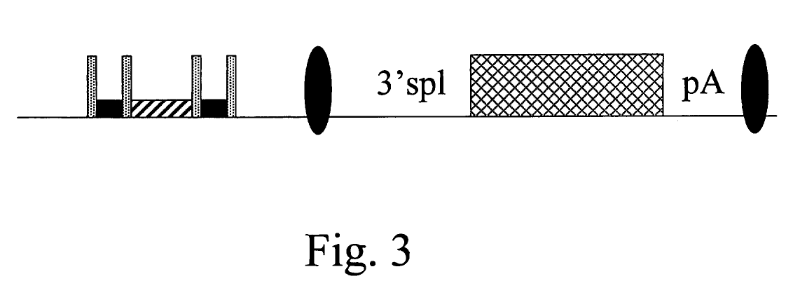

In one embodiment the targeting construct comprises a lac OCR element (two lac operators spaced 150 bp apart) separated by a 400 bp spacer sequence, comprising the rabbit β-globin second intron, followed by another lac OCR element (See FIG. 3). Immediately 3′(downstream) of the lacO elements is a loxP-flanked cassette consisting of a 3′splice site and an internal ribosome entry site (IRES, for translation initiation) linked to a GFPneo fusion sequence with its own poly(A) addition sequence (See FIG. 3). This insertion vector is designed for random mutagenesis of endogenous genes. Multiple lacO binding sites ensures that operator-bound lac repressor will be able to block transcription elongation, while the 3′splice site is designed for trapping the construct within an intron (splicing should occur between the 5′splice site of the intron and the 3′splice site provided by the construct). Thus only when the construct is inserted into an intron will the marker gene undergo post-transcriptional modification and become operably linked to the coding region of endogenous gene

In one embodiment the marker gene comprises a GFPneo cassette. This cassette includes a GFP reporter that is sensitive to incorporation into an active transcription unit, and is also a selectable marker for positive selection of transfected ES clones with G418. The reporter also allows characterization of randomly targeted ES cell clones for their ability to be regulated by lac repressor. In the presence of lac repressor, expression of the GFPneo cassette will be suppressed, while in the presence of IPTG, removal of the lac repressor-mediated block of transcription elongation should result in GFPneo expression. The loxP sites allow Cre-mediated excision of the reporter sequences. This is necessary so that expression from the lacO-targeted gene in the absence of lac repressor is not truncated at the polyA site associated with the GFPneo cassette.

The operator targeting construct of the present invention can be formulated as part of a kit that is used to produce transgenic organisms. In one embodiment the kit comprises two gene constructs for preparing transgenic mammals for in vivo regulation of gene expression. The first construct comprises a eukaryotic promoter linked to the modified repressor gene of the present invention and the second construct comprises the operator targeting construct of the present invention. In particular, the operator targeting construct comprises an operator sequence operably linked to a reporter gene construct, wherein the reporter gene construct comprises a 3′ splice acceptor sequence and a reporter gene, and the reporter gene construct is flanked at either end by direct repeats of a site specific recombinase target sequence. In one preferred embodiment the operator targeting construct has the structure shown in FIG. 3, wherein the operator sequence is selected from the group consisting of SEQ ID NO: 5, SEQ ID NO: 6, SEQ ID NO: 19 or SEQ ID NO: 20.

In accordance with the present invention a transgenic animal can be prepared using the operator targeting construct of the present invention. The method comprises the steps of introducing the targeting construct into the cell of the plant or animal using standard transgenic techniques, identifying those plants or animals that are expressing the reporter gene, and introducing site-specific recombinase activity to remove the reporter gene cassette. In one embodiment the site-specific recombinase activity is introduced by inducing the expression of a recombinase gene already present in the animal or plant. Alternatively the recombinase activity can be introduced into the plant/animals progeny by crossing the original transgenic (or it progeny) with a transgenic line that constitutively expresses recombinase activity in its cells. The resultant transgenic organisms, comprising a targeted insertion of lacO elements within an intron should be capable of conferring multiple rounds of both gene repair and inactivation under the control of the lac repressor.

A transgenic animal comprising a gene operably linked to an operator and a repressor gene construct (a “double transgenic”) is then created by introducing a repressor encoding nucleic acid sequence into an animal (or its progeny) that comprises a gene operably linked to an operator. In one embodiment the step of creating the double transgenic animal comprises mating a transgenic animal comprising the operator-containing endogenous gene with a transgenic animal that comprises a repressor gene construct of the present invention. In one embodiment the repressor gene construct comprises a eukaryotic promoter operably linked to the sequence of SEQ ID NO: 4.

The creation of conditional alleles and transgenes that can be switched on and off reversibly will enable studies relating to the reversibility of the disease processes. Such models could become important tools for evaluating the consequences of silencing or reactivating the expression of normal or mutant genes on disease progression, and for determining the efficacy of potential therapeutic strategies that can be applied even after overt symptoms of a disease have developed.

### EXAMPLE 1

### Target Gene Activity is Controlled by LacI and IPTG in the Mouse

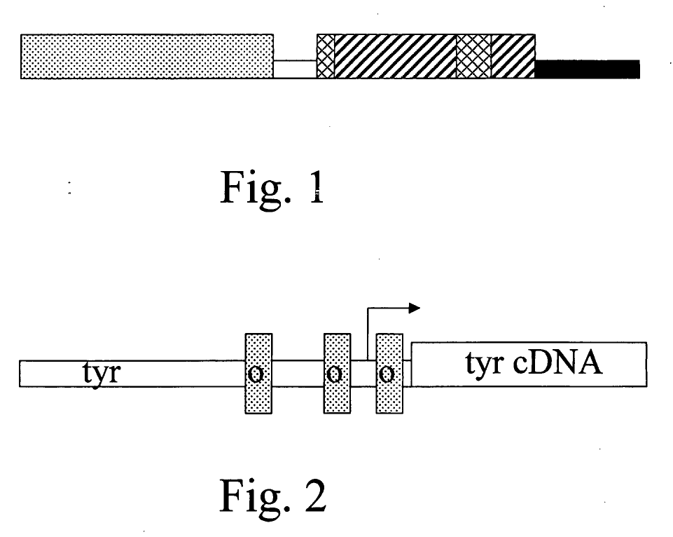

The lac operator-repressor system of the present invention was tested in mice using a regulatable version of a well-characterized visible marker gene, tyrosinase. Tyrosinase is the protein product of the albino (c) locus (Kwon et al., PNAS 84, 7473-7477 (1987)), and is the enzyme that catalyzes the first step in melanin biosynthesis. The target transgene consists of the wildtype murine tyrosinase cDNA under the control of the murine tyrosinase promoter modified to contain lac operator sequences (See FIG. 2). The major transcription start site in the tyrosinase promoter is 83 bp upstream of the start codon. To maintain the endogenous spacing of promoter elements in the critical region between the start of transcription and the start of translation, a PCR-based, site-directed mutagenesis was used to change 25 bp of the endogenous sequence to create a primary lac operator centered at 59 bp upstream of the start of translation. Additional operators were inserted 176 bp and 526 bp upstream of the primary operator (FIG. 2). Mice containing this modified Tyrosinase transgene resemble pigmented animals previously described (Methot et al., nucleic Acids Research, 23, 4551-4556 (1995)) that had been microinjected with an unregulatable version of the same transgene. Two lines of pigmented Tyrosinase transgenic mice were established containing the regulatable transgene. The Tyr^{lacO} (25) line displays a himalayan pigmentation pattern, and the Tyr^{lacO} (43) displays a light pigmentation pattern, similar to those described in Methot et al.(1995). Mice transgenic for the Tyrosinase transgene were crossed to mice transgenic for LacI.

In double transgenics, the lac repressor should bind to the operator sequences located in the tyrosinase promoter, block transcription of tyrosinase, and revert pigmented animals to albino. This was in fact observed in the double transgenic mice. The coat of the double transgenic is unpigmented and indistinguishable from that of a nontransgenic albino. Treatment of a double transgenic animal with 10 mM IPTG in the drinking water derepressed tyrosinase expression, resulting in a phenotype indistinguishable from that of the mouse transgenic for Tyr^{lacO} construct alone. The stringency of repression and derepression was evident from observation of the pigmentation of the eye. Comparison of sections through the eyes of a nontransgenic albino, a Tyr transgenic, a Tyr, LacI double transgenic, and a Tyr, LacI double transgenic mouse treated with IPTG revealed that repression of the target tyrosinase transgene expression is accompanied by an absence of melanin in the retinal pigment epithelium (RPE) of the double-transgenic animal. The entire RPE is devoid of melanin, as can be seen by the completely unpigmented appearance of the eye in whole mount. Derepression by IPTG is accompanied by a restoration of pigmentation in the RPE to levels indistinguishable from the nonrepressed state.

Similar results were obtained with both lines of regulatable Tyrosinase mice. This indicates that regulation is neither insertion site specific nor simply fortuitous, but rather controlled by the lac repressor acting specifically on a target gene with lac operator sequences in its promoter. The albino mutation is a single base pair change in the coding sequence of tyrosinase, which causes a single amino acid change in the protein. Because the mutant allele is both transcribed and translated, promoter activity has not been assayed quantitatively at the molecular level. Nevertheless, one can infer from its effect on pigmentation that the tyrosinase promoter is in fact regulated by the lac operator-repressor system tightly, in a biologically relevant manner.

These results also show that IPTG can be introduced into the drinking water and circulate in the mouse at a level sufficient to derepress target gene expression. This level appears to be completely nontoxic. Tyr^{lacO}, LacI double-transgenic mice have been administered 10 mM IPTG in their drinking water for up to 8 months with no deleterious effects.

### Materials and Methods

Construction of lac Repressor Genes

The lac repressor constructs W, S, 5′C1, 5′C2, 5′C4, 3′C1, 3′C2, 3′C3, and 3′C4 are driven by a 4.3-kb promoter region from the human β-actin gene from the Eco RI site up to the AluI site at −7 (Leavitt et al. 1984). Followed by a short linker of either 44 bp (gatcagtcga cctgcagcccaagcttgata tcgaattcgg atct; SEQ ID NO: 13) in W, 5′C1, 5′C4, 3′C1, 3′C2, 3′C3, and 3′C4 or 30 bp (gatcagtcga cctgcagccc aagcttcacc; SEQ ID NO: 14) in S and 5′C2. 5′C3 contains the 4.3-kb promoter up to the start of translation with no polylinker. All of the above constructs contain the polyadenylation signal sequence from the bovine growth hormone gene (Woychik et al. 1982) connected to the 3′ end of the construct by a Bam HI and Eco RI linker region (taggatccccgggctgcagg aattc; SEQ ID NO: 15).

Coding regions for the original wtlacI (W) and synlacI (S) constructs are as previously described (Scrable and Stambrook 1997). 5′C1 and 5′C2 were made by switching the linker region and the first 36 bp of the coding region between wtlacI and synlacI using the BsrFI site shared by both constructs. 5′C1 contains the wtlacI linker and first 36 bp of the coding region, and then the synlacI coding region. The nuclear localization signal sequence (NLS) that had been attached to the synlacI coding sequence was removed by PCR mutagenesis, so that 5′C1 codes for a protein identical in amino-acid sequence to the endogenous lac repressor. 5′C2 contains the synlacI linker and first 36 bp of the coding region, and then the 3′ wtlacI coding region through the stop site. 5′C3 is identical to the endogenous β-actin promoter up to the ATG start site, then contains the original synlacI coding region. This was created by PCR mutagenesis to remove the linker region present in S and replace the 6 bp missing between the AluI site and +1. 5′C4 contains the linker region from W and the entire synlacI coding region, with no NLS. This was created by PCR mutagenesis of 5′C1 to return the four bases in the beginning of the coding region that differ between W and S back to the synlacI sequence. 3′C1 contains the wtlacI sequence from the start of translation up to the EcoRV site at +800 (which W and S have in common) and the synlacI sequence after the EcoRV site. The SV40 NLS is attached to the 3′ end of the 3′C1 coding region with the linker region (agcagcctgaggcct; SEQ ID NO: 16), as described (Fieck et al. Nucleic Acid Res, 20, 1785-1791 (1992)), and was created by PCR mutagenesis of the existing NLS linker region described in Scrable and Stambrook (1997). 3′C2 is identical to 5′C1 up to the EcoRV site, then identical to W downstream. 3′C3 is identical to 5′C1 up to the PvuII site at +950 from the start of translation, then identical to W downstream. 3′C4 is identical to W upstream of the PvuII site, then identical to 3′C1 downstream.

Constructs M and R contain the human B-actin promoter blunted at the AscI site at 70 followed by the rabbit B-globin intron 2 from the blunted NcoI site through the EcoRI site in exon 3. The lacI coding region is inserted at the EcoRI site. The M coding region is identical to W, and the R coding region is identical to 3′C4. The rabbit β-globin fragment continues downstream of the lacI coding region to include the rest of exon 3 and the β-globin 3′ untranslated region with a polyadenylation signal sequence. The polyA signal sequence from SV40 also is present at the 3′ end. All of the B-globin sequences and the SV40 polyA signal sequence are as described (Katsuki et al. Science 271, 1247-1254 (1988)).

RT-PCR Assay for Splice Site Use in lac Repressor Transcripts

Total RNA was extracted from testis of W and S transgenic animals, or Rat 2 fibroblasts transfected by calcium phosphate with the indicated lacI construct using TRI Reagent (Molecular Research Products, Inc.). RNA was DNase treated with RQ1 DNase (Promega) and 1 μg reverse transcribed with AMV-RT. cDNA was subjected to 30 rounds of amplification (95° C. for 30 sec, 60° C. for 30 sec, 72° C. for 1 min.) using a primer in the β-actin promoter (acagagcctcgcctttg; SEQ ID NO: 17) and a primer in the lacI coding sequence (tgcaggcagcttccaca; SEQ ID NO: 18). PCR products were run on a 4% polyacrylamide gel in 1×TBE and transferred to Hybond-N+ membrane (Amersham) by semi-dry electrophoresis in NAQ transfer solution (0.08 M Tris-HCl, 0.118 M Borate, 2.4 mM EDTA, pH8.3) at 220 mA for 1 h. The resultant Southern blot was UV crosslinked and then prehybridized and hybridized according to the methods described in Scrable and Stambrook (1997).

Rat2 Transfection Assay for lac Repressor Function

Rat 2 fibroblasts were transfected with 2.5 μg pSVOZ DNA (pSVOZ is a construct comprising the SV40 early promoter that contains a single, symmetrical operator driving the expression of the β-galactosidase (lacZ) reporter gene, which contains the endogenous O_{z} operator) and 2.5 μg of the indicated lac repressor construct DNA (or pBSSK carrier DNA) per 3×10⁵ cells by standard calcium phosphate-mediated transfection. Growth media was DMEM, 0.1 units/mL penicillin/0.1 μg/mL streptomycin (Life Technologies), 5% FCS (Hyclone); (with 20 mM IPTG, if indicated). Two days after transfection, cells were fixed in 0.5% gluteraldehyde, incubated with X-gal containing solution (0.5 mg X-gal in dimethylformamide, 44 mM HEPES, 3 mM potassium ferrocyanide, 3 mM potassium ferricyanide, 1.5 mM NaCl, 0.13 mM MgCl₂ at pH>7) at 37° C. overnight, and the number of blue cells in each well recorded.

Nucleic Acid Extraction and Blotting

Northern blots were prepared as described in Scrable and Stambrook (1997), except that RNA was transferred to Biodyne A nylon membrane. DNA was extracted from tail biopsies using the simplified-method as described in Laird et al. Nucleic Acid Res 19, 4293 (1991). Southern blots were prepared and analyzed according to the methods given in Scrable and Stambrook (1997).

Detection of lac Repressor Protein by Western Blot and Immunohistochemistry

A panel of monoclonal antibodies to the lac repressor was created by injecting a LacI-TrpE fusion protein into mice. For Western blots, total protein was extracted into lysis solution (50 mM Tris at pH 7.5, 0.15 M NaCl, 1% Nonidet P40), containing protease inhibitors (0.25% sodium deoxycholate, 1 mM PMSF, 2 mM EGTA, 1 μM leupeptin, 0.2 μM aprotinin, 0.8 mM N-ethylmalemide, 2 μM pepstatin A). Protein concentration was determined by Lowry's assay, and 30 μg run on a 12% SDS-PAGE gel. The proteins were transferred to nitrocellulose membrane with semi-dry electrophoresis, and blocked in 5% dried milk in PBS overnight. The blot was incubated with biotinylated anti-lacI antibody 5F8 (25 μg/mL in 1% BSA/TBST) for 1 h at 37° C., labeled with peroxidase (ABC reagent, Vector) and visualized with chemiluminscence (SuperSignal, Pierce) on a ChemiImager (Alpha Innotech Corp.).

For immunohistochemistry, mice were given a lethal dose of Nembutol sodium, and perfused with 4% paraformaldehyde for 30 min. Tissues were placed in 20% sucrose overnight at 4° C., frozen, sectioned at 30 μm, and thaw-mounted onto Superfrost Plus (Fisher) slides. Sections were incubated with biotinylaed anti-lacI antibody 9A5 (3 μg/mL in 1% BSA/0.3% Triton-X100 in PBS) overnight at 4° C., labeled with peroxidase (ABC reagent, Vector), and visualized with DAB.

Construction of the Regulatable Tyrosinase Transgene (Tyr^{lacO})

The regulatable Tyr^{lacO} transgene is based on the construct TYBS described in Yokoyama et al. Nucleic Acids Res. 18, 7293-7298 (1990). The first lac operator was created by site-directed mutagenesis (ExSite, Stratagene). 25 bp of the endogenous promoter sequence (from 72 to 48) was changed to make a 29 bp operator centered at 59, identical in sequence to the primary operator of the lac operon (gtggaattgt gagcggataa caatttcac; SEQ ID NO: 19) (Lewis et al.1996). Two additional operators with the same sequence were inserted as part of a 47 bp fragment (agatctgtgg aattgtgagc ggataacaat ttcacggatc cagatct; SEQ ID NO: 20) into the BsrGI site at 203 and the EcoRV site at 548 of the promoter.

Production of Transgenic Mice

Production of the W and S lines is described in Scrable and Stambrook (1997). The rest of the transgenic lines described were produced by microinjection into the outbred ICR line (Harlan) using standard procedures. Two transgenic founders were made for the 3′C4 transgene; both showed a testis-only expression pattern. One founder line was established for the M construct. Three founders were transgenic for R; two (lines 1 and 3) exhibited ubiquitous expression, and one (line 13) had more limited expression that ranged from low to moderate in various tissues. Eight founders were transgenic for Tyr^{lacO}; an F1 generation was produced from all eight, and two of those established pigmented transgenic lines (lines 25 and 43). Of the animals indicated as Tyrosinase transgenic, two were homozygous for Tyr^{lacO}, and all others were hemizygous for Tyr^{lacO}. All lacI transgenic mice described were hemizygous for lacI.

Analysis of Eye Pigmentation and IPTG Treatment of Mice

For adult eyes, mice were given a lethal dose of Nembutol sodium, perfused transcardially (1.25% paraformaldehyde, 1.5% gluteraldehyde, in 0.1 M phosphate at pH 7.4); eyes were dissected out and photographed. They then were embedded in parafin, sectioned at 10 μm, dewaxed in Xylene, hydrated in decreasing concentrations of ethanol, and reacted in cresyl violet (0.5% in 20% ethanol, pH to 2.5 with glacial-acetic acid) for 8 min, dehydrated, cleared, and mounted in DPX. For embryonic eyes, pregnant females were euthanized at E12.5, and the embryos removed. The head of each embryo was fixed in 2% paraformaldehyde in 0.1 M phosphate (pH 7.4), and the lower half was taken for genotyping.

Tyrosinase activity was derepressed by replacing the drinking water with a 10 mM solution of IPTG (changed every four days). To allow for the normal pattern of tyrosinase expression during development, the female was started on 10 mM IPTG at day 7 of pregnancy.

### Example 2

### Regulation is Functional During Embryogenesis, and Reversible

To determine if lac elements could regulate pigmentation during embryogenesis and if IPTG could act transplacentally, the pigmentation in the developing retinal pigment epithelium of embryonic and newborn mice was investigated. At E9, tyrosinase activity in the embryonic eye of wild type mice begins to deposit pigment in the developing retinal pigment epithelium. At E12.5, the mouse RPE clearly is pigmented. Tyr^{lacO} (43) transgenic mice recapitulate this developmental event. A distinct band of pigmentation surrounding the central lens is seen in the Tyrosinase transgenic embryo that is not seen in the nontransgenic albino. The lac repressor blocked pigmentation during embryogenesis in the double-transgenic embryo, but not when the mother was treated with IPTG during pregnancy.

These results clearly demonstrate not only that lac regulatory sequences function well during embryogenesis, but also that IPTG can cross the placenta to alter the phenotype of developing animals. Finally, the reversibility of the system was tested by switching the Tyrosinase transgene on after it had been off, or off after it had been on, in the same animal. The phenotypes of eyes of newborn mice were compared to the phenotypes of embryonic eyes of mice of the same genotype. When the mother of the IPTG-treated Tyr^{lacO}, LacI double transgenic was not started on IPTG in her drinking water until E12.5 of the pregnancy fully pigmented eyes were seen in the progeny at birth. However, at E12.5, this double transgenic pup has the albino phenotype. The fully pigmented eyes seen at birth in this animal demonstrate that even after a period of silencing, derepression by IPTG was able to switch tyrosinase expression on. Reversibility was also observed with regards to switching off the tyrosinase gene after it had intially been on. When a Tyr^{lacO}, LacI double-transgenic pup's mother was taken off IPTG at P9, removal of IPTG caused reversion of the coat phenotype to albino. As expected, the eyes remain pigmented due to the low turnover of cells and melanosomes in the RPE.

In summary, by modifying both the target promoter and the gene encoding the lac repressor, a regulatory system used in bacteria was successfully adapted to control the transcription of a gene so that it can function analogously in the complex environment of the mouse. The LacI mouse described in this report expresses the lac repressor ubiquitously, so it can be used in the future to regulate other promoters with the same degree of control demonstrated for the Tyrosinase transgene. In addition, it is anticipated that endogenous promoters can be targeted for insertion of operator elements into the endogenous promoters through homologous recombination. This would move the system to the next level, where endogenous loci can be switched on and off repeatedly to create reversible models of human disease and normal development in the mouse.

### Materials and Methods

Preparation of Primary Mouse Embryo Cells

Pregnant females were euthanized on day E13.5 (where E0.5 was the day a vaginal plug was observed). The embryos were dissected out and a small section frozen for genotyping. Embryonic tissue was minced and placed in 2-mL dissociation solution [2 mg/mL Collagenase B, 2 U/mL RQ1 DNase in RPMI 1640 media (GIBCO)] at 37° C. for 2 h, triturating the solution after 1 h. Cells were spun at 175 g, washed one time with Hank's BSS, plated with growth media [DMEM, 0.1 units/mL penicillin/0.1 μg/mL streptomycin (GIBCO), 10% FCS (Hyclone)], and transfected by calcium phosphate.

Analysis of Eye Pigmentation and IPTG Treatment of Mice

For embryonic eyes, pregnant females were euthanized at E12.5, and the embryos removed. The head of each embryo was fixed in 2% paraformaldehyde in 0.1 M phosphate (pH 7.4), and the lower half was taken for genotyping. Tyrosinase activity was derepressed by replacing the drinking water with a 10 mM solution of IPTG (changed every four days). To allow for the normal pattern of tyrosinase expression during development, the female was started on 10 mM IPTG at day 7 of pregnancy.

1. A nucleic acid sequence comprising the sequence of SEQ ID NO: 1.

2. The nucleic acid sequence of claim 1 further comprising a nuclear localization signal.

3. The nucleic acid sequence of claim 2 wherein the sequence comprises the sequence of SEQ ID NO: 2 or SEQ ID NO: 4.

4. The nucleic acid sequence of claim 2 further comprising
a mammalian promoter that comprises a transcriptional start site; and
an intron, wherein said promoter is operably linked to said intron and said intron is operably linked to the 5′ end of sequence of SEQ ID NO: 2, wherein said intron provides adequate spacing so that two 100 bp regions located approximately 600 and 800 bp downstream of the transcription start site are devoid of CpG dinucleotides.

5. The nucleic acid sequence of claim 3 wherein said sequence comprises the sequence of SEQ ID NO: 4.

6. The nucleic acid sequence of claim 5 further comprising a promoter operably linked to the sequence of SEQ ID NO: 4.

7. The nucleic acid construct of claim 6 formed as a plasmid.

8. A host cell comprising the nucleic acid construct of claim 2.

9. The host cell of claim 8 wherein the cell is a eukaryotic cell.

10. The cell of claim 9 wherein said construct is inserted into the genome of the cell.

11. A non-human transgenic mammal comprising an exogenous DNA molecule that is stably integrated in its genome, wherein said exogenous DNA molecule comprises the nucleic acid sequence of claim 4.

12. The transgenic mammal of claim 11 wherein the exogenous DNA molecule comprises a mammalian promoter operably linked to the nucleic acid sequence of SEQ ID NO: 4.

13. The transgenic animal of claim 12 further comprising a nucleic acid sequence that comprises an operator operably linked to a gene.

14. A kit for regulating the expression of a gene, said kit comprising
a first nucleic acid sequence comprising the sequence of claim 4; and
a second nucleic acid sequence comprising an operator operably linked to a promoter.

15. The kit of claim 14 wherein the first nucleic acid sequence comprises a promoter operably linked to the sequence of SEQ ID NO: 4; and the second nucleic acid sequence further comprises a polylinker operably linked to the promoter of said second nucleic acid sequence.

16. The kit of claim 15 wherein said fist and second nucleic acid sequences are formed as plasmids.

17. A method of regulating the expression of a gene in a transgenic animal, said method comprising the steps of
providing a transgenic animal wherein the cells of said animal comprise a first nucleic acid sequence comprising the sequence of claim 4, and a second nucleic acid sequence comprising an operator operably linked to said gene;
contacting the cells of said animal in vivo with an inducer of said repressor.

18. The method of claim 17 wherein said first nucleic acid sequence comprises a mammalian promoter operably linked to the sequence of SEQ ID NO: 4.

19. The method of claim 18 wherein the step of contacting the cells comprises administering said inducer orally or intraperitoneal.

20. A method of regulating the expression of an endogenous gene in vivo said method comprising the steps of
providing a transgenic animal that comprises an operator sequence inserted into an endogenous gene; and
introducing a repressor encoding nucleic acid sequence, comprising a eukaryotic promoter operably linked to the sequence of SEQ ID NO: 4, into that transgenic animal or its progeny.

21. The method of claim 20, wherein the operator sequence inserted into an endogenous gene by homologous recombination after a nucleic acid sequence comprising the operator sequence is introduced into a cell of said animal.

22. The method of claim 21 wherein the step of introducing the repressor encoding nucleic acid sequence comprises mating a transgenic animal comprising the operator containing endogenous gene with a transgenic animal that comprises the nucleic acid sequence comprising a eukaryotic promoter operably linked to the sequence of SEQ ID NO: 4.

23. An operator targeting construct comprising
an operator sequence;
two direct repeats of a site specific recombinase target sequence; and
a reporter gene construct, said reporter gene construct comprising a reporter gene and a 3′ splice acceptor site located upstream from said reporter gene, wherein the operator is operably linked to the reporter gene construct and said reporter gene construct is flanked by direct repeats of the site-specific recombinase target sequence.

24. The construct of claim 23 wherein the operator sequence is selected from the group consisting of SEQ ID NO: 5 or SEQ ID NO: 6;
the site specific recombinase target sequence is a loxP site or an FRT site.

25. The construct of claim 24 wherein the operator targeting construct further comprises a second operator separated from the first operator by about 150 to about 200 base pairs of DNA.


SEQUENCE LISTING (20 sequences)

SEQ ID NO: 1 - DNA, 1080 bp, Artificial Sequence
  This sequence represents a modified E. coli lac repressor gene
atgaaaccagtaacgttatacgatgtcgcagagtatgccggtgtctcttatcagactgtttccagagtggtgaaccaggccagccatgtttctgccaaaaccagggaaaaagtggaagcagccatggcagagctgaattacattcccaacagagtggcacaacaactggcaggcaaacagagcttgctgattggagttgccacctccagtctggccctgcatgcaccatctcaaattgtggcagccattaaatctagagctgatcaactgggagcctctgtggtggtgtcaatggtagaaagaagtggagttgaagcctgtaaagctgcagtgcacaatcttctggcacaaagagtcagtgggctgatcattaactatccactggatgaccaggatgccattgctgtggaagctgcctgcactaatgttccagcactctttcttgatgtctctgaccagacacccatcaacagtattattttctcccatgaagatggtacaagactgggtgtggagcatctggttgcattgggacaccagcaaattgcactgcttgcgggcccactcagttctgtctcagcaaggctgagactggccggctggcataaatatctcactaggaatcaaattcagccaatagctgaaagagaaggtgactggagtgccatgtctgggtttcaacaaaccatgcaaatgctgaatgagggcattgttcccactgcaatgctggttgccaatgatcagatggcactgggtgcaatgagagccattactgagtctgggctgagagttggtgcagatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgccagtctcactggtgaagagaaaaaccaccctggcacccaatacacaaactgcctctccccgggcattggctgattcactcatgcagctagcaagacaggtttccagactggaaagtgggcag

SEQ ID NO: 2 - DNA, 1119 bp, Artificial Sequence
  This sequence represents a modified E. coli lac repressor gene (same as SEQ ID NO 1) with a 39bp SV40 localization signal located at the C-terminus
atgaaaccagtaacgttatacgatgtcgcagagtatgccggtgtctcttatcagactgtttccagagtggtgaaccaggccagccatgtttctgccaaaaccagggaaaaagtggaagcagccatggcagagctgaattacattcccaacagagtggcacaacaactggcaggcaaacagagcttgctgattggagttgccacctccagtctggccctgcatgcaccatctcaaattgtggcagccattaaatctagagctgatcaactgggagcctctgtggtggtgtcaatggtagaaagaagtggagttgaagcctgtaaagctgcagtgcacaatcttctggcacaaagagtcagtgggctgatcattaactatccactggatgaccaggatgccattgctgtggaagctgcctgcactaatgttccagcactctttcttgatgtctctgaccagacacccatcaacagtattattttctcccatgaagatggtacaagactgggtgtggagcatctggttgcattgggacaccagcaaattgcactgcttgcgggcccactcagttctgtctcagcaaggctgagactggccggctggcataaatatctcactaggaatcaaattcagccaatagctgaaagagaaggtgactggagtgccatgtctgggtttcaacaaaccatgcaaatgctgaatgagggcattgttcccactgcaatgctggttgccaatgatcagatggcactgggtgcaatgagagccattactgagtctgggctgagagttggtgcagatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgccagtctcactggtgaagagaaaaaccaccctggcacccaatacacaaactgcctctccccgggcattggctgattcactcatgcagctagcaagacaggtttccagactggaaagtgggcagagcagcctgaggcctcccaagaagaagcgaaaggtgtga

SEQ ID NO: 3 - protein, 372 aa, Escherichia coli
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPSTTIKQDFRLLGQTSVDRLLQLSQGQAVKGNQLLPVSLVKRKTTLAPNTQTASPRALADSLMQLARQVSRLESGQSSLRPPKKKRKV

SEQ ID NO: 4 - DNA, 2163 bp, Artificial Sequence
  This sequence represents a modified E. coli lac repressor gene with a 39bp SV40 localization signal (same as SEQ ID NO 2) but with E.coli B-galactosidase noncoding regions located bp 1-505 and at bp 1625-2163
catggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagatgaaaccagtaacgttatacgatgtcgcagagtatgccggtgtctcttatcagactgtttccagagtggtgaaccaggccagccatgtttctgccaaaaccagggaaaaagtggaagcagccatggcagagctgaattacattcccaacagagtggcacaacaactggcaggcaaacagagcttgctgattggagttgccacctccagtctggccctgcatgcaccatctcaaattgtggcagccattaaatctagagctgatcaactgggagcctctgtggtggtgtcaatggtagaaagaagtggagttgaagcctgtaaagctgcagtgcacaatcttctggcacaaagagtcagtgggctgatcattaactatccactggatgaccaggatgccattgctgtggaagctgcctgcactaatgttccagcactctttcttgatgtctctgaccagacacccatcaacagtattattttctcccatgaagatggtacaagactgggtgtggagcatctggttgcattgggacaccagcaaattgcactgcttgcgggcccactcagttctgtctcagcaaggctgagactggccggctggcataaatatctcactaggaatcaaattcagccaatagctgaaagagaaggtgactggagtgccatgtctgggtttcaacaaaccatgcaaatgctgaatgagggcattgttcccactgcaatgctggttgccaatgatcagatggcactgggtgcaatgagagccattactgagtctgggctgagagttggtgcagatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgccagtctcactggtgaagagaaaaaccaccctggcacccaatacacaaactgcctctccccgggcattggctgattcactcatgcagctagcaagacaggtttccagactggaaagtgggcagagcagcctgaggcctcccaagaagaagcgaaaggtgtgaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatcttattaaagcag

SEQ ID NO: 5 - DNA, 30 bp, Escherichia coli
tgtggaattgtgagcgctcacaattccaca

SEQ ID NO: 6 - DNA, 18 bp, Escherichia coli
attgtgagcgctcacaat

SEQ ID NO: 7 - protein, 7 aa, Simian virus 40
PKKKRKV

SEQ ID NO: 8 - protein, 5 aa, Adenovirus type 37
KRPRP

SEQ ID NO: 9 - DNA, 27 bp, Escherichia coli
atgaaaccagtaacgttatacgatgtc

SEQ ID NO: 10 - DNA, 39 bp, Artificial Sequence
  Synthetic sequence used to link the repressor coding sequence to the localization signal sequence
agcagcctgaggcctcccaagaagaagcgaaaggtgtga

SEQ ID NO: 11 - DNA, 2294 bp, Artificial Sequence
  This sequence represents a modified E. coli lac repressor gene with a 39bp SV40 localization signal and E.coli B-galactosidase noncoding regions (same as SEQ ID NO 4) with a polyA signal sequence from SV40 located at bp 2164-2294
catggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagatgaaaccagtaacgttatacgatgtcgcagagtatgccggtgtctcttatcagactgtttccagagtggtgaaccaggccagccatgtttctgccaaaaccagggaaaaagtggaagcagccatggcagagctgaattacattcccaacagagtggcacaacaactggcaggcaaacagagcttgctgattggagttgccacctccagtctggccctgcatgcaccatctcaaattgtggcagccattaaatctagagctgatcaactgggagcctctgtggtggtgtcaatggtagaaagaagtggagttgaagcctgtaaagctgcagtgcacaatcttctggcacaaagagtcagtgggctgatcattaactatccactggatgaccaggatgccattgctgtggaagctgcctgcactaatgttccagcactctttcttgatgtctctgaccagacacccatcaacagtattattttctcccatgaagatggtacaagactgggtgtggagcatctggttgcattgggacaccagcaaattgcactgcttgcgggcccactcagttctgtctcagcaaggctgagactggccggctggcataaatatctcactaggaatcaaattcagccaatagctgaaagagaaggtgactggagtgccatgtctgggtttcaacaaaccatgcaaatgctgaatgagggcattgttcccactgcaatgctggttgccaatgatcagatggcactgggtgcaatgagagccattactgagtctgggctgagagttggtgcagatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgccagtctcactggtgaagagaaaaaccaccctggcacccaatacacaaactgcctctccccgggcattggctgattcactcatgcagctagcaagacaggtttccagactggaaagtgggcagagcagcctgaggcctcccaagaagaagcgaaaggtgtgaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatcttattaaagcagtaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 12 - DNA, 277 bp, Artificial Sequence
  This represents a 276 bp fragment of the cloning vector pBR327 (from the BamHI site at bp 375 to the SalI site at bp 651).
ggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcg

SEQ ID NO: 13 - DNA, 44 bp, Escherichia coli
gatcagtcgacctgcagcccaagcttgatatcgaattcggatct

SEQ ID NO: 14 - DNA, 30 bp, Artificial Sequence
  Synthetic nucleic acid sequence linking the promoter to the coding region
gatcagtcgacctgcagcccaagcttcacc

SEQ ID NO: 15 - DNA, 25 bp, Artificial Sequence
  Synthetic nucleic acid sequence linking the promoter to the coding region
taggatccccgggctgcaggaattc

SEQ ID NO: 16 - DNA, 15 bp, Artificial Sequence
  Synthetic nucleic acid sequence linking the coding region to the polyadenylation sequence
agcagcctgaggcct

SEQ ID NO: 17 - DNA, 17 bp, Artificial Sequence
  PCR primer for the beta-actin promoter
acagagcctcgcctttg

SEQ ID NO: 18 - DNA, 17 bp, Artificial Sequence
  PCR primer for the lacI coding sequence
tgcaggcagcttccaca

SEQ ID NO: 19 - DNA, 29 bp, Escherichia coli
gtggaattgtgagcggataacaatttcac

SEQ ID NO: 20 - DNA, 47 bp, Escherichia coli
agatctgtggaattgtgagcggataacaatttcacggatccagatct